(19) United States
(12) Patent Application Publication
(10) Pub. No.: US 2023/0027480 A1
(43) Pub. Date: Jan. 26, 2023

(54) MEASUREMENT OF THERAPEUTIC PROTEINS CO-ADMINISTERED TO A SUBJECT BY LC-MRM-MS ASSAY

(71) Applicant: Regeneron Pharmaceuticals, Inc., Tarrytown, NY (US)

(72) Inventors: Xuefei Zhong, White Plains, NY (US); Yuan Mao, Hartsdale, NY (US)

(21) Appl. No.: 17/716,154

(22) Filed: Apr. 8, 2022

Related U.S. Application Data
(60) Provisional application No. 63/224,952, filed on Jul. 23, 2021.
(60) Provisional application No. 63/172,567, filed on Apr. 8, 2021.

(51) Int. Cl. G01N 33/68; G01N 30/72; C12Q 1/37

(52) U.S. Cl. CPC G01N 33/6848; G01N 30/7233; G01N 33/6854; C12Q 1/37; G01N 2560/00; G01N 2030/8831

(57) ABSTRACT

The present invention generally pertains to methods of quantitating therapeutic proteins co-administered to a subject using LC-MRM-MS. In particular, the present invention pertains to the use of dual enzymatic digestion to generate unique surrogate peptides allowing for the accurate quantitation of co-administered therapeutic proteins using LC-MRM-MS.

### CROSS-REFERENCE TO RELATED APPLICATIONS

This application claims priority to and the benefit of U.S. Provisional Patent Application No. 63/172,567, filed Apr. 8, 2021 and U.S. Provisional Patent Application No. 63/224,952, filed Jul. 23, 2021 which are each herein incorporated by reference.

### FIELD

This application relates to assay methods for the quantitation of one or more therapeutic proteins co-administered to a subject.

### BACKGROUND

Liquid chromatography coupled to tandem mass spectrometry (LC-MS/MS) is becoming a preferred method for analysis of biopharmaceuticals, such as therapeutic proteins. While ligand binding assays (LBAs) have conventionally been used for this purpose, LC-MS/MS offers a number of advantages that provide for a much faster method development process. A key aspect of using LC-MS/MS to analyze a therapeutic protein is through quantitation of a surrogate peptide, derived from proteolytic digestion, as a unique identifier of the protein.

Therapeutic proteins may be administered to a subject individually or may be co-administered, for example, in an antibody cocktail. In this case, interference from matrix components or competition from co-administered therapeutic proteins may make it difficult to identify and quantitate a unique surrogate peptide for a therapeutic protein of interest, and therefore to quantitate said therapeutic protein of interest, or multiple therapeutic proteins of interest.

Therefore, it will be appreciated that a need exists for methods to accurately, rapidly and simultaneously quantitate multiple co-administered therapeutic proteins.

### SUMMARY

A liquid chromatography-multiple reaction monitoring mass spectrometry (LC-MRM-MS) based approach combined with dual enzymatic digestion was developed for determination of total concentrations of each antibody component of an antibody cocktail in serum samples. The performance characteristics of this bioanalytical assay were evaluated with respect to linearity, accuracy, precision, selectivity, specificity, and analyte stability before and after enzymatic digestion. The developed LC-MRM-MS assay has a dynamic range from about 10 to 2000 μg/mL of antibody drug in human serum matrix, which was able to cover the serum drug concentration from Day 0 to Day 28 after drug administration in two dosage groups for clinical pharmacokinetic study. The pharmacokinetic profiles in two dosage groups measured by the MRM assay were comparable to those measured by fully validated electrochemiluminescence (ECL) immunoassays.

This disclosure provides a method for simultaneously quantitating at least two co-administered therapeutic proteins. In some exemplary embodiments, the method comprises (a) obtaining a sample including a first therapeutic protein and a second therapeutic protein; (b) generating a unique surrogate peptide for each of said first and second therapeutic proteins by contacting said sample to at least two digestive enzymes; (c) quantitating said surrogate peptides using a mass spectrometer; and (d) quantitating said first and second therapeutic proteins using the quantitated surrogate peptides.

In one aspect, said digestive enzymes are chosen from a group consisting of trypsin, chymotrypsin, LysC, LysN, AspN, GluC and ArgC. In a specific aspect, said digestive enzymes comprise trypsin and AspN.

In one aspect, said first and second therapeutic proteins comprise an antibody, a monoclonal antibody, a bispecific antibody, an antibody fragment, a Fab region of an antibody, an antibody-drug conjugate, or a fusion protein. In another aspect, said first therapeutic protein comprises casirivimab and said second therapeutic protein comprises imdevimab.

In one aspect, said mass spectrometer is an electrospray ionization mass spectrometer, nano-electrospray ionization mass spectrometer, or a triple quadrupole mass spectrometer. In another aspect, said mass spectrometer is coupled to a chromatography system. In a specific aspect, said chromatography system comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said sample comprises human serum. In another aspect, said method further comprises selecting said digestive enzymes using in silico analysis of potential surrogate peptides. In yet another aspect, said quantitation of surrogate peptides comprises the use of multiple reaction monitoring. In a further aspect, said method further comprises administering said first and second therapeutic proteins to a subject.

In one aspect, said method has a dynamic range of about 10 to about 2000 μg/mL of the first therapeutic protein in the sample. In another aspect, said method has a dynamic range of about 10 to about 2000 μg/mL of the second therapeutic protein in the sample. In yet another aspect, said mass spectrometer is capable performing a multiple reaction monitoring or parallel reaction monitoring.

In one aspect, said method further comprises the steps of conducting peptide mapping of said surrogate peptides, selecting unique peptides and fragment ions of the surrogate peptides to generate multiple reaction monitoring transitions, selecting the top two or top three transitions of the surrogate peptides, optimizing collision energy of the surrogate peptides, subsequently generating a calibration curve, and determining a LLOQ (lower limit of quantification) according to the calibration curve.

In one aspect, said method further comprises selecting said at least one surrogate peptide specific to said first or said second therapeutic protein, wherein the at least one surrogate peptide is pre-selected by determining that (i) the surrogate peptide is specific to a digest of the therapeutic protein to be quantified; (ii) the surrogate peptide is specifically absent from the protease digest of the preparation in the absence of the at least one therapeutic protein; (iii) the surrogate peptide produces a strong signal in a mass-spectrographic analysis; and (iv) the surrogate peptide produces a distinguishable signal in a mass-spectrographic analysis.

In one aspect, said method further comprises denaturing said first therapeutic protein and said second therapeutic protein prior to step (b). In another aspect, said denaturing comprises contacting said first therapeutic protein and said second therapeutic protein to a denaturation solution. In a further aspect, said denaturation solution comprises tris(2-carboxyethyl)phosphine hydrochloride (TCEP-HCl), urea, or a combination thereof. In another specific aspect, said denaturing comprises heating said sample to about 80° C.

In one aspect, said method further comprises reducing said first therapeutic protein and said second therapeutic protein prior to step (b). In another aspect, said reducing comprises contacting said first therapeutic protein and said second therapeutic protein to a reduction agent. In a further aspect, said reduction agent is tris(2-carboxyethyl)phosphine hydrochloride (TCEP-HCl).

In one aspect, said method further comprises alkylating said first therapeutic protein and said second therapeutic protein prior to step (b). In another aspect, said alkylating comprises contacting said first therapeutic protein and said second therapeutic protein to an alkylating agent. In a further aspect, said alkylating agent is iodoacetamide.

This disclosure also provides a method for simultaneously quantitating casirivimab and imdevimab from an administered antibody cocktail. In some exemplary embodiments, the method comprises (a) obtaining a serum sample including casirivimab and imdevimab; (b) generating at least one unique surrogate peptide for each of casirivimab and imdevimab by contacting said sample to trypsin and AspN; (c) quantitating said surrogate peptides using a mass spectrometer; and (d) quantitating casirivimab and imdevimab using the quantitated surrogate peptides.

In one aspect, said surrogate peptides comprise the amino acid sequences LLIYAASNLETGVPSR and DTAVYYCASGS. In another aspect, said mass spectrometer is an electrospray ionization mass spectrometer, nano-electrospray ionization mass spectrometer, or a triple quadrupole mass spectrometer. In yet another aspect, said mass spectrometer is coupled to a liquid chromatography system.

In one aspect, said method further comprises administering casirivimab and imdevimab to a subject. In another aspect, said mass spectrometer is capable performing a multiple reaction monitoring or parallel reaction monitoring.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates a workflow for a LC-MRM-MS/MS assay according to an exemplary embodiment.

FIG. 2A shows a collision-induced dissociation (CID) MS/MS spectrum of a surrogate peptide for mAb1 generated from trypsin and AspN digestion according to an exemplary embodiment. FIG. 2B shows a CID MS/MS spectrum of a surrogate peptide for mAb2 generated from trypsin and AspN digestion according to an exemplary embodiment.

FIG. 3 shows extracted ion chromatograms (XICs) of surrogate peptides (FIG. 3A, FIG. 3D), internal standards (FIG. 3B, FIG. 3E) and calibration curve plots (FIG. 3C, FIG. 3F) of mAb1 (FIGS. 3A-C) and mAb2 (FIGS. 3D-F) generated from LC-MRM-MS of calibration standards according to an exemplary embodiment.

FIG. 4A shows overlaid XICs of a surrogate peptide of mAb1 from ten individual naive human serum samples co-spiked with 10 μg/mL of mAb1 and 20 μg/mL of mAb2 according to an exemplary embodiment. FIG. 4B shows overlaid XICs of a surrogate peptide of mAb2 from ten individual naive human serum samples co-spiked with 10 μg/mL of mAb1 and 20 μg/mL of mAb2 according to an exemplary embodiment. FIG. 4C shows a measured accuracy percentage of drug concentrations in the ten individual human serum samples with drugs spiked at lower limit of quantitation (LLOQ) level according to an exemplary embodiment.

FIG. 5A shows XICs of the MRM transition for the surrogate peptide of mAb1 according to an exemplary embodiment. FIG. 5B shows XICs of the MRM transition for the surrogate peptide of mAb2 according to an exemplary embodiment. FIG. 5C shows a comparison of the accuracy percentage of drug concentrations of mAb1 at five quality control (QC) levels measured without the presence of mAb2 in serum matrix or with 2 mg/mL mAb2 in the serum matrix background according to an exemplary embodiment. FIG. 5D shows a comparison of the accuracy percentage of drug concentrations of mAb2 at five QC levels measured without the presence of mAb1 in serum matrix or with 2 mg/mL mAb1 in the serum matrix background according to an exemplary embodiment.

FIG. 6A shows the accuracy percentage of measuring mAb1 stability in three different conditions at five QC levels according to an exemplary embodiment. FIG. 6B shows the accuracy percentage of measuring mAb2 stability in three different conditions at five QC levels according to an exemplary embodiment.

FIG. 7A shows the pharmacokinetic profile of mAb1 measured from serum samples by the LC-MRM-MS assay and a fully validated electrochemiluminescence (ECL) immunoassay according to an exemplary embodiment. FIG. 7B shows the pharmacokinetic profile of mAb2 measured from serum samples by the LC-MRM-MS assay and a fully validated ECL immunoassay according to an exemplary embodiment.

### DETAILED DESCRIPTION

REGEN-COV (casirivimab and imdevimab) is an investigational antibody cocktail therapy developed by Regeneron Pharmaceuticals, Inc. for the treatment of coronavirus disease 2019 (COVID-19) caused by severe acute respiratory syndrome coronavirus 2 (SARS-CoV-2) (Hansen et al., 2020, Science, 369:1010-1014; Baum et al., 2020, Science, 370:1110-1115; Weinreich et al., 2021, N Engl J Med, 384:238-251). The antibody cocktail includes two humanized IgG1 monoclonal antibodies (herein referred to as mAb1 and mAb2), which are designed to target non-overlapping epitopes on the SARS-CoV-2 spike protein, and thereby blocking the interaction of SARS-CoV-2 virus with human ACE2, and preventing viral escape due to rapid genetic mutation of the virus (Hansen et al.; Baum et al., 2020, Science, 369:1014-1018). A recent clinical study has shown that REGEN-COV therapy can reduce viral load and improve symptoms for non-hospitalized COVID-19 patients, especially those who were seronegative or had high viral loads at baseline (Weinrich et al.). Based on the promising results from the clinical investigation, REGEN-COV was granted Emergency Use Authorization (EUA) by the U.S. Food and Drug Administration (FDA) in November 2020 for the treatment of recently diagnosed, mild-to-moderate COVID-19 in adults and pediatric patients at least 12 years of age and weighing at least 40 kg who are at high risk for progressing to severe COVID-19 and/or hospitalization.

Measurement of the time profile of antibody drug concentration in serum after drug administration in patients is critical for pharmacokinetic (PK) characterization of protein therapeutic and drug dose optimization. To meet this need and manage the accelerated development for a COVID-19 therapy, a fit-for-purpose liquid chromatography-multiple reaction monitoring mass spectrometry (LC-MRM-MS) assay for REGEN-COV pharmacokinetic study was developed and qualified in one month, a much shorter timeframe than that required for the development of a conventional ligand-binding assay. Unlike a ligand-binding assay, an LC-MRM-MS assay does not require highly specific affinity capture and detection reagents for the antibody therapeutics, which typically take several months to develop and produce. In addition, the LC-MRM-MS assay of the present invention also provides wide dynamic range, good accuracy and precision, and excellent selectivity and specificity for quantification of protein-based biopharmaceuticals in serum matrix (van den Broek et al., 2013, J Chromatogr B, 929:161-179). Recently, LC-MRM-MS has become a more frequently adopted bioanalytical strategy for both preclinical and clinical sample analysis due to the continuous improvement on the performance of LC-MS instrumentation (Jiang et al., 2013, Anal Chem, 85:9859-9867; Zhang et al., 2014, Anal Chem, 86:8776-8784; Li et al., 2012, Anal Chem, 84:1267-1273; Cardozo et al., 2020, Nat Commun, 11:6201; Fernandez Ocana et al., 2012, Anal Chem, 84:5959-5967; Shen et al., 2015, Anal Chem, 87:8555-8563).

Quantification of total antibody drug concentration, including free and bound antibodies, in human serum samples using LC-MRM-MS can be based on the measurement of ion intensities of the surrogate peptides derived from the variable complementarity-determining regions (CDRs) of the antibody drugs (Jenkins et al., 2015, AAPS J, 17:1-16). To process patient serum samples, typically a few microliters of serum sample was reduced, alkylating, and then underwent protease digestion. Stable heavy isotope labeled proteins or surrogate peptides are usually used as internal standards (ISs) to normalize the signal variation from sample processing and instrument performance fluctuation. The sensitivity, selectivity and specificity of the assay can rely on the unique CDR peptides that have been selected for quantification. For a co-administered antibody cocktail, the LC-MRM-MS can be readily multiplexed to measure multiple drug analytes simultaneously. Despite limited throughput due to the chromatographic separation, the LC-MRM-MS method of the present invention met the required dynamic range, sensitivity, selectivity, stability, and specificity for the early measurement of drug concentrations of REGEN-COV in a limited number of serum samples in clinical trials. The concentrations of REGEN-COV in two dose groups of ambulatory patients measured by the LC-MRM-MS assay of the invention were compared with the results obtained from a fully validated ligand binding immunoassay, which demonstrated that the two assays were in good agreement. This disclosure sets an example as a fit-for-purpose application of LC-MRM-MS for clinical sample analysis when there are challenges to deliver a validated immunoassay to meet an urgent timeline, or if high-quality anti-idiotypic antibody reagents for a ligand binding assay are not available.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. A protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, a protein of interest can be a recombinant protein, an antibody, a bispecific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG (e.g., IgG1, IgG2, IgG3, IgG4), IgM, IgA1, IgA2, IgD, or IgE. In certain exemplary embodiments the antibody molecule is a full-length antibody (e.g., an IgG1 or IgG4 immunoglobulin) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Muller & Roland E. Kontermann, Bispecific Antibodies, HANDBOOK OF THERAPEUTIC ANTIBODIES 265-310 (2014), the entire teachings of which are herein incorporated).

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, a protein of interest can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK' (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, the term “therapeutic protein” refers to any protein that can be administered to a subject for the treatment of a disease or disorder. A therapeutic protein may be any protein with a pharmacological effect, for example, an antibody, a soluble receptor, an antibody-drug conjugate, or an enzyme. In some exemplary embodiments, the therapeutic protein can be an anti-SARS-CoV-2 antibody, including casirivimab or imdevimab. Multiple therapeutic proteins may be co-administered in order to achieve a pharmacological effect, for example, to prevent viral escape due to mutation of a target virus. As used herein, the term “antibody cocktail” refers to co-administered therapeutic proteins comprising at least two therapeutic antibodies. In some exemplary embodiments, an antibody cocktail can comprise REGEN-COV.

In some exemplary embodiments, the number of therapeutic proteins in the sample can be at least two. In some specific embodiments, one of the therapeutic proteins can be a monoclonal antibody, a polyclonal antibody, a bispecific antibody, an antibody fragment, a fusion protein, or an antibody-drug complex. In some other specific embodiments, a concentration of one of the therapeutic proteins in a sample can be about 10 μg/mL to about 2000 μg/mL. In some exemplary embodiments, the number of therapeutic proteins in the sample is three. In some exemplary embodiments, the number of therapeutic proteins in the sample is four. In some exemplary embodiments, the number of therapeutic proteins in the sample is five.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, viral inactivation, or filtration.

In some exemplary embodiments, the sample is a biological sample. As used here, the term “biological sample” refers to a sample taken from a living organism, for example a human or a non-human mammal. A biological sample may comprise, for example, whole blood, plasma, serum, saliva, tears, semen, cheek tissue, organ tissue, urine, feces, skin, or hair. A sample may be taken from a patient, for example, a clinical sample.

As used herein, the term “pharmacokinetics” (PK) refers to a field of study dealing with features of a drug after administration to a subject. Exemplary components of pharmacokinetic analysis include liberation of a drug from a pharmaceutical formulation, absorption of a drug into blood circulation, distribution of a drug throughout the body, metabolism (also called biotransformation) of a drug into metabolites, and excretion of a drug from a body. Pharmacokinetics of a drug are a key feature for evaluation of a biotherapeutic candidate. In particular, a pharmacokinetic study may be conducted to evaluate how levels of a drug and its modified forms and metabolites change over time after administration to a subject. Biotherapeutic proteins may be evaluated through the analysis of representative peptides, or “target peptides” or “surrogate peptides,” using liquid chromatography-mass spectrometry. A peptide may be a suitable target peptide if it is unique to or strongly representative of a protein, for example a complementarity-determining region of an antibody, and if it can be reliably recovered and measured.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography.

As used herein, the term “mass spectrometer” includes a device capable of identifying specific molecular species and measuring their accurate mass-to-charge ratios. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion, fragmenting it (MS2), isolating a primary fragment ion, fragmenting it (MS3), isolating a secondary fragment, and so on (MS4), as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some exemplary embodiments, the mass spectrometer can be coupled to a liquid chromatography system.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring—based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some aspects, the mass spectrometer in the method or system of the present application can be an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-MRM-MS (liquid chromatography-multiple reaction monitoring-mass spectrometry) analyses.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

The amount of digestive enzyme and the time required for digestion can be appropriately selected. When the enzyme to substrate ratio is unsuitably high, the correspondingly high digestion rate will not allow sufficient time for the peptides to be analyzed by mass spectrometer, and sequence coverage will be compromised. On the other hand, a low enzyme to substrate ratio would need a long digestion time and thus a long data acquisition time. The enzyme to substrate ratio can range from about 1:0.5 to about 1:200.

In some exemplary embodiments, the method of quantitating a therapeutic protein can optionally comprise contacting a therapeutic protein to a protein reducing agent.

As used herein, the term “protein reducing agent” refers to the agent used for reduction of disulfide bridges in a protein. Non-limiting examples of protein reducing agents are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In some exemplary embodiments, the method of quantitating protein can optionally comprise contacting a therapeutic protein to a protein alkylating agent.

As used herein, the term “protein alkylating agent” refers to the agent used to alkylate certain free amino acid residues in a protein. Non-limiting examples of protein alkylating agents are iodoacetamide (IAM), chloroacetamide (CAA), acrylamide (AA), N-ethylmaleimide (NEM), methyl methanethiosulfonate (MMTS), and 4-vinylpyridine or combinations thereof.

In some exemplary embodiments, the method of quantitating a therapeutic protein can comprise denaturing a therapeutic protein.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

It is understood that the present invention is not limited to any of the aforesaid therapeutic protein(s), antibody cocktail(s), host cell(s), protein denaturing agent(s), protein alkylating agent(s), protein reducing agent(s), digestive enzyme(s), mass analyzer(s), instrument(s) used for identification, or chromatographic method(s), and any therapeutic protein(s), antibody cocktail(s), host cell(s), protein denaturing agent(s), protein alkylating agent(s), protein reducing agent(s), digestive enzyme(s), mass analyzer(s), instrument(s) used for identification, or chromatographic method(s) can be selected by any suitable means.

The present invention will be more fully understood by reference to the following Examples. They should not, however, be construed as limiting the scope of the invention.

### EXAMPLES

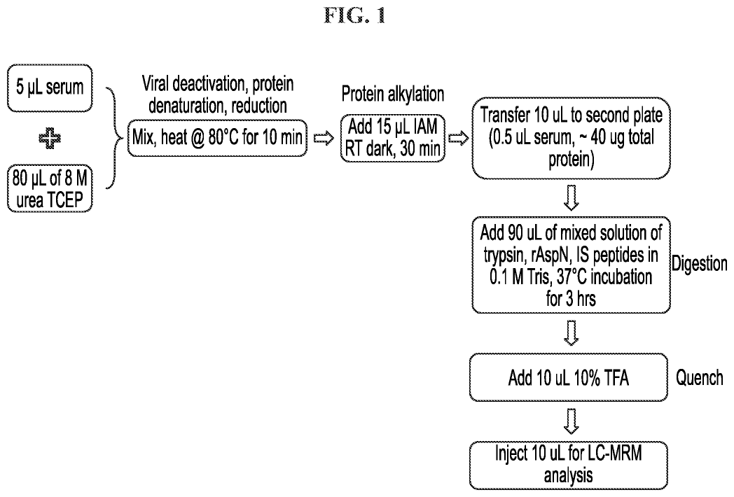

The overall workflow of the LC-MRM-MS/MS assay of the invention according to an exemplary embodiment is illustrated in FIG. 1.

Chemicals and reagents. Tris (2-carboxyethyl) phosphine hydrochloride (TCEP-HCl), trifluoroacetic acid (TFA), 0.1% formic acid (v/v) in water (LC-MS grade), and 0.1% formic acid (v/v) in acetonitrile (LC-MS grade) were purchased from Thermo Fisher Scientific (Rockford, IL). Ultrapure 1 M Tris-HCl pH 8.0 was obtained from Invitrogen (Carlsbad, Calif.). Urea and iodoacetamide (IAM) were purchased from Sigma-Aldrich (St. Louis, Mo.). Trypsin (Mass Spectrometry grade) and rAspN were purchased from Promega (Madison, Wis.). Pooled human serum and single human serum from 10 individuals were purchased from Innovative Research (Novi, Mich.). AUQA grade custom synthetic heavy peptides for internal standards (ISs), LLIYAASNLETGVPSR*(10 Da), DTAV*(6Da) YYCASGS, were ordered from Thermo Fisher Scientific (Rockford, Ill.). mAb1 and mAb2 drug substance (DS) were developed and obtained from Regeneron Pharmaceuticals (Tarrytown, N.Y.). COVID-19 patient serum samples were from a clinical trial of REGEN-COV sponsored by Regeneron Pharmaceuticals (ClinicalTrials.gov Identifier: NCT04425629).

Preparation of standard solutions. Stock solutions of REGEN-COV in human serum were made by spiking mAb1 and mAb2 DS into pooled human serum. Calibration standards (20, 25, 30, 50, 100, 250, 500, 1000, 2000 μg/mL for mAb1; 10, 20, 25, 30, 50, 100, 250, 500, 1000, 2000 μg/mL for mAb2) were made through a serial dilution of the stock solution using the pooled human serum. Five qualification QC standards including the Upper Limit of Quantitation (ULOQ, 2000 μg/mL mAb1, 2000 μg/mL mAb2), High QC (HQC, 1500 μg/mL mAb1, 1500 μg/mL mAb2), Mid QC (MQC, 750 μg/mL mAb1, 750 μg/mL mAb2), Low QC (LQC, 60 μg/mL mAb1, 30 μg/mL mAb2), and the Lower Limit of Quantitation (LLOQ, 20 μg/mL mAb1, 10 μg/mL mAb2), were also prepared by spiking mAb1 and mAb2 DS into the pooled human serum and serial dilutions.

LLOQ (20 μg/mL of mAb1, 10 μg/mL of mAb2) spiked individual human serum samples were prepared by co-spiking mAb1 and mAb2 DS into ten individual human serum blanks. For the drug specificity assay, the QC standards containing one drug were made by serial dilution of stock solution of the antibody drug using the pooled human serum as the diluent. The QC standards containing one drug with the presence of co-administered drug as matrix background were made from serial dilution of the stock solution using 2 mg/mL of the co-administered drug in the pooled human serum as the diluent.

Digestion of serum samples. Prior to sample processing, serum samples (calibration standards, QC standards, and patient samples) were thawed on ice. The serum sample digestion was conducted in a 96-well plate (0.5 mL, polypropylene, Agilent Technologies, Santa Clara, Calif.). 5 μL of serum sample was added to each sample well prefilled with 80 μL denaturation solution (10 mM TCEP, 8 M urea). The 96-well plate was sealed with an adhesive plate seal (Waters, Milford, Mass.), and heated at 80° C. for 10 minutes on Thermomixer C (Eppendorf, Hamburg, Germany) at 650 rpm. After cooling to room temperature, 15 μL of 0.25 M IAM was added to each sample well and the plate was incubated by shaking at 650 rpm in dark for 30 minutes at room temperature. Prior to use, digestion solution containing two enzymes and two IS peptides were made by reconstitution of 200 μg of trypsin, 100 μg of rAspN, 150 μL of mAb1 IS stock solution (5 pmol/μL), and 100 μl of mAb2 IS stock solution (5 pmol/μL) in 9 mL of 0.1 M Tris buffer. Following alkylation, 10 μL of each sample were transferred to a second 96-well plate and mixed with 90 μL digestion solution containing two enzymes and IS peptides. The sample plate was sealed and incubated at 37° C. for 3 hours with 650 rpm shaking. When the digestion finished, 10 μL of 10% TFA was added to each sample well to quench the reaction. The sample plate was spun at 700 rpm for 1 minute prior to LC-MRM-MS analysis.

LC-MRM-MS methods. The LC-MRM-MS experiments were performed using an Agilent Infinity II UPLC system coupled with 6495 Triple Quadrupole Mass Spectrometer (Agilent Technologies, Santa Clara, Calif.). 10 μL of digested serum sample, corresponding to approximately 45 nL of original serum, were loaded onto a C18 column (ACQUITY UPLC BEH300 1.7 μm, 2.1 mm×100 mm, Waters), and separated by reversed phase gradient elution using mobile phase A as 0.1% formic acid in water, and mobile phase B as 0.1% formic acid in acetonitrile at flow rate of 0.3 mL/min. Prior to each injection, the sample injection path was sequentially flushed with IPA/ACN/H₂O v/v/v (3:1:1), ACN/H₂O/FA v/v/v (25:75:0.1), and ACN/H₂O/FA v/v/v (5:95:0.1). The LC gradient for MRM experiments was set as follows: 0-0.5 min, 5% B; 0.5-16 min, 5-25% B; 16-18 min, 25˜90% B; 18-20 min, 90% B; 20-20.5 min, 90˜5% B, and 20.5˜25, 5% B. The column temperature was set at 60 ° C. and the autosampler was maintained at 7° C. during sample analysis.

The triple quadrupole MS ion source parameters were set as follows: gas temperature 200° C., gas flow rate 12 L/min, nebulizer gas 20 psi, sheath gas temperature 300° C., sheath gas flow 11 L/min, capillary voltage 3500 V, nozzle voltage 500 V. Time scheduled MRM transitions for the two surrogate peptides and two IS peptides, with parameters of each transition channel listed in Table 1-1 and Table 1-2, were applied for all the quantitative analysis experiments.

TABLE 1-1
Time scheduled MRM transitions for
LC-MRM-MS data acquisition

| | | | Precursor | Product | Precursor | Product |
|---|---|---|---|---|---|---|
| Time | Drug | Peptide | Charge | Type | Ion | Ion |
| 3.0-10.0 min | mAb2 | DTAVYYC | 2 | y⁵⁺ | 597.6 | 481.2 |
| | | (Cam)ASGS | | | | |
| | | DTAV*(6Da) | 2 | y⁵⁺ | 600.6 | 487.2 |
| | | YYC(Cam)AS | | | | |
| | | GS | | | | |
| 10.0-16.0 min | mAb1 | LLIYAASNLE | 2 | y³⁺ | 853.0 | 359.2 |
| | | TGVPSR | | | | |
| | | LLIYAASNLE | 2 | y³⁺ | 858.0 | 369.2 |
| | | TGVPSR* | | | | |
| | | (10Da) | | | | |

TABLE 1-2
Time scheduled MRM transitions
for LC-MRM-MS data acquisition

| | | | | | Cell |
|---|---|---|---|---|---|
| | | Dwell | | | Accelerator |
| Time | Drug | Time (ms) | Fragmentor | CE | Voltage |
| 3.0-10.0 min | mAb2 | 300 | 380 | 6 | 5 |
| | | 300 | 380 | 6 | 5 |
| 10.0-16.0 min | mAb1 | 300 | 380 | 18 | 5 |
| | | 300 | 380 | 18 | 5 |

Data analysis. The raw data from LC-MRM-MS experiments were analyzed using Agilent MassHunter Quantitative Analysis software. The extracted ion chromatogram (XIC) peak areas of the monitored transitions were integrated with the Agile2 algorithm. To construct the calibration curve for each drug, the peak areas of surrogate peptide from calibration standards, normalized by the peak areas from the corresponding coeluting IS peptide, were plotted against their respective nominal concentrations using a 1/x² weighted three parameter quadratic model (with variable weight for each point of the standard curve), from which all other readings were subsequently calculated. The equation for quadratic fit is y=ax²+bx+c, where y=ratio of the XIC peak area of the surrogate peptide and that of the corresponding IS peptide, x=concentration of drug (μg/mL), and a, b, c=quadratic coefficient, linear coefficient and constant term, respectively. The weight for each point of the standard curve is inversely proportional to the analyte concentration. The calibration curve parameters were automatically computed by Agilent MassHunter Quantitative Analysis software.

Electrochemiluminescent immunoassay. The assay procedures employed streptavidin microplates coated with either biotinylated mouse anti-mAb1 monoclonal antibody, or biotinylated mouse anti-mAb2 monoclonal antibody. mAb1 and mAb2 captured on plates specific for each molecule were detected using two ruthenylated, non-competing mouse monoclonal antibodies that are specific to either mAb1 or mAb2. Electrochemiluminescent signal generated from the ruthenium label when voltage is applied to the plate was measured by the MSD reader. The measured electrochemiluminescence is proportional to the concentration of total mAb1 or total mAb2 in the serum samples.

### Example 1

### Method Development for MRM Assay

Both mAb1 and mAb2 are dimer molecules composed of a pair of light chains and a pair of heavy chains. mAb1 light chain comprises 221 amino acid residues, and its heavy chain comprises 450 amino acid residues. mAb2 light chain comprises 216 amino acid residues, and its heavy chain comprises 450 amino acid residues. Because IgG proteins are abundant in human serum, and greater than 93% of the amino acid sequences of these two humanized IgG1 drugs are identical to the endogenous serum IgG, the selection of suitable surrogate peptides for MRM-based IgG antibody drug quantification is restricted to peptides derived from the CDR regions (typically 3 from the heavy chain, 3 from the light chain) of the variable domains. Peptides from the constant regions cannot be differentiated from those derived from endogenous antibodies in human serum.

To select suitable surrogate peptides for MRM quantification, the following considerations were applied to screen the candidate peptides generated by protease cleavage in the CDR region of the human IgG drugs: 1) no identical BLAST match hit in Uniprot human proteome database (www.uniprot.org/blast/); 2) peptide length shorter than 20 amino acid residues; 3) sequence does not contain sites prone to missed cleavages during enzymatic digestion, such as KR, RDR for trypsin cleavage; and 4) sequence does not contain sites susceptible to in vivo biotransformation or residues prone to partial modification during sample processing, such as methionine. By applying these criteria to examine the in silico trypsin digestion generated CDR peptides of mAb1 and mAb2, it was found that only one peptide, LLIYAASNLETGVPSR, which is from the light chain CDR2 of mAb1, could serve as the surrogate peptide for mAb1 quantification. None of the tryptic peptides from mAb2 CDR regions could satisfy all of the criteria listed above, as shown in Table 2-1. In this case, another protease, rAspN, was used to generate a unique surrogate peptide with appropriate length from the heavy chain CDR3, DTAVYYCASGS, for mAb2 quantification, as shown in Table 2-2. CDR region sequences are indicated in bold letters. The reasons for excluding the peptide as an MRM surrogate peptide for MRM method development are marked with “x”.

TABLE 2-1
Prediction of peptide sequences
containing CDRs by trypsin
digestion of mAb1 and mAb2.

| | | | Peptide | Identical | |
|---|---|---|---|---|---|
| | | | length | sequence | PTM |
| | CDR | Peptide | shorter | match by | mod |
| | region | sequence | than 20 | BLAST | (Met) |
| mAb1 | HC_CDR1 | LSCAASGFTFSD | | X | X |
| | | YYMSWIR | | | |
| | HC_CDR2 | GLEWVSYITYSG | X | | |
| | | STIYYADSVK | | | |
| | HC_CDR3 | AEDTAVYYCAR | | X | |
| | HC_CDR3 | GTTMVPFDYWGQ | | | X |
| | | GTLVTVSSASTK | | | |
| | LC_CDR1 | VTITCQASQDIT | X | | |
| | | NYLNWYQQKPGK | | | |
| | LC_CDR2 | LLIYAASNLETG | | | |
| | | VPSR | | | |
| | LC_CDR3 | FSGSGSGTDFTF | X | | |
| | | TISGLQPEDIAT | | | |
| | | YYCQQYDNLPLT | | | |
| | | FGGGTK | | | |
| mAb2 | HC_CDR1 | LSCAASGFTFSN | | | X |
| | | YAMYWVR | | | |
| | HC_CDR2 | GLEWVAVISYDG | | X | |
| | | SNK | | | |
| | HC_CDR3 | TEDTAVYYCASG | X | | |
| | | SDYGDYLLVYWG | | | |
| | | QGTLVTVSSAST | | | |
| | | K | | | |
| | LC_CDR1 | QSALTQPASVSG | X | X | |
| | | SPGQSITISCTG | | | |
| | | TSSDVGGYNYVS | | | |
| | | WYQQHPGK | | | |
| | LC_CDR2 | LMIYDVSK | | X | |
| | LC_CDR3 | SGNTASLTISGL | X | | |
| | | QSEDEADYYCNS | | | |
| | | LTSISTWVFGGG | | | |
| | | TK | | | |

TABLE 2-2
Prediction of peptide sequences
containing CDRs by combined trypsin
and AspN digestion of mAb2.

| | | | Peptide | Identical | |
|---|---|---|---|---|---|
| | | | length | sequence | PTM |
| | CDR | Peptide | shorter | match by | mod |
| | region | sequence | than 20 | BLAST | (Met) |
| mAb2 | HC_CDR1 | LSCAASGFTFSN | | | X |
| | | YAMYWVR | | | |
| | HC_CDR2 | GLEWVAVISY | | X | |
| | HC_CDR2 | DGSNK | | X | |
| | HC_CDR3 | DTAVYYCASGS | | | |
| | HC_CDR3 | DYLLVYWGQGTL | X | | |
| | | VTVSSASTK | | | |
| | LC_CDR1 | QSALTQPASVSG | X | X | |
| | | SPGQSITISCTG | | | |
| | | TSS | | | |
| | LC_CDR1 | DVGGYNYVSWYQ | | X | |
| | | QHPGK | | | |
| | LC_CDR2 | DVSK | | X | |
| | LC_CDR3 | DYYCNSLTSIST | X | | |
| | | WVFGGGTK | | | |

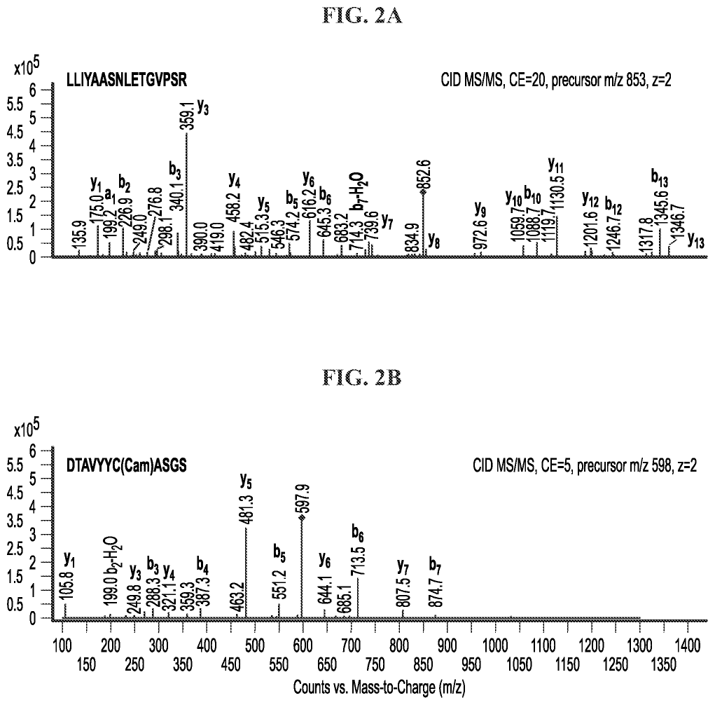

The trypsin digests of mAb1 drug substance and rAspN digests of mAb2 drug substance were used to optimize the MRM transition parameters on an Agilent QQQ system. Time scheduled product ion scan experiments for the surrogate peptide candidates during reverse phase LC separation were performed to select the best transition and collisional energy. Based on the CID MS/MS spectra acquired, the transition from the +2 precursor ion to y3 product ion was selected to monitor the abundance of the mAb1 surrogate peptide LLIYAASNLETGVPSR (FIG. 2A); and transition from the +2 precursor ion to y5 product ion was selected to monitor the abundance of the alkylated mAb2 surrogate peptide DTAVYYC(Cam)ASGS (FIG. 2B). Notably, the optimal collisional energy for this doubly charged mAb2 surrogate peptide is about 5 V, which is much smaller compared to the typical collisional energy required for doubly charged tryptic peptides.

The selected transition channels of the surrogate peptides, and their corresponding transitions for internal standard peptides, were examined for human serum matrix background interference. Pooled human serum blank, as well 10 individual serum blank samples (5 female, 5 male), digested with a combination of trypsin and rAspN, were analyzed with a 16 minute reversed phase LC gradient and time scheduled MRM acquisition of the four transition channels. Signal interference was not observed from either the light or heavy transition channels of the two surrogate peptides. After evaluation of the matrix interference of the selected transitions for the two surrogate peptides, the instrument parameters were further optimized under MRM mode, and the parameters listed in Table 1-1 and Table 1-2 were applied for assay qualification and patient sample analysis.

### Example 2

### LC-MRM-MS Assay Qualification

Prior to application on clinical sample analysis, the performance of the developed LC-MRM-MS assay was evaluated using the following most critical parameters: 1) linearity, 2) accuracy and precision, 3) selectivity, 4) specificity, and 5) analyte stability before and after sample digestion.

### 2.1 Linearity

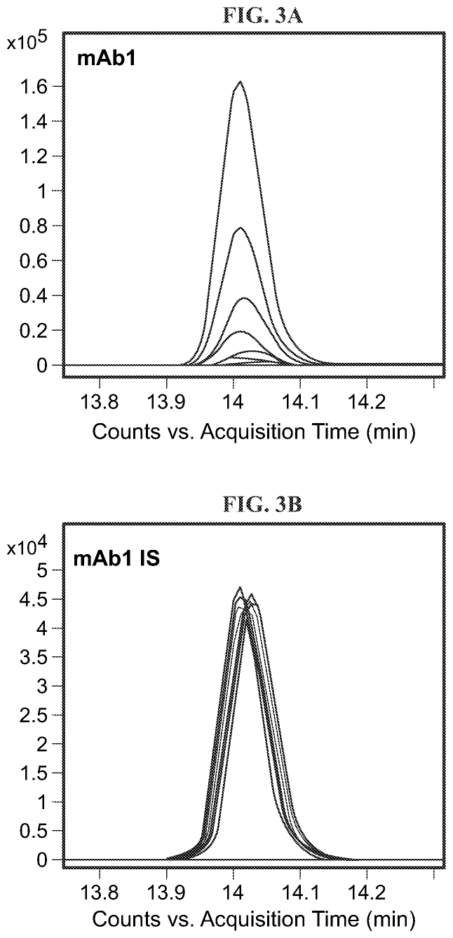

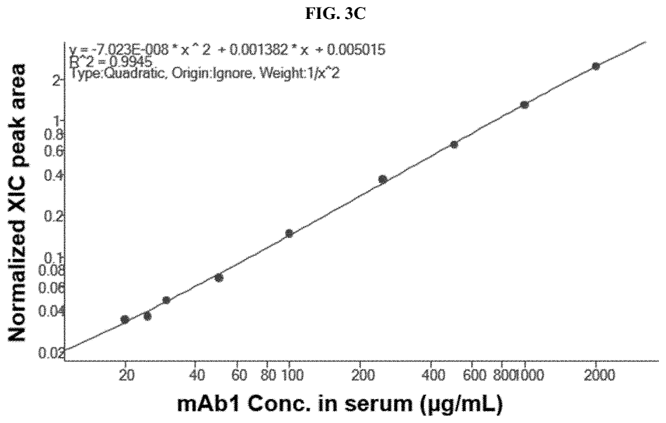

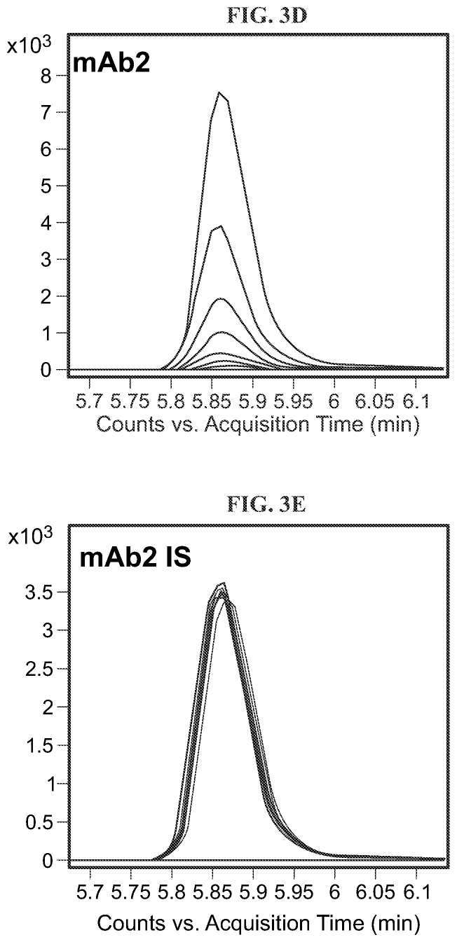

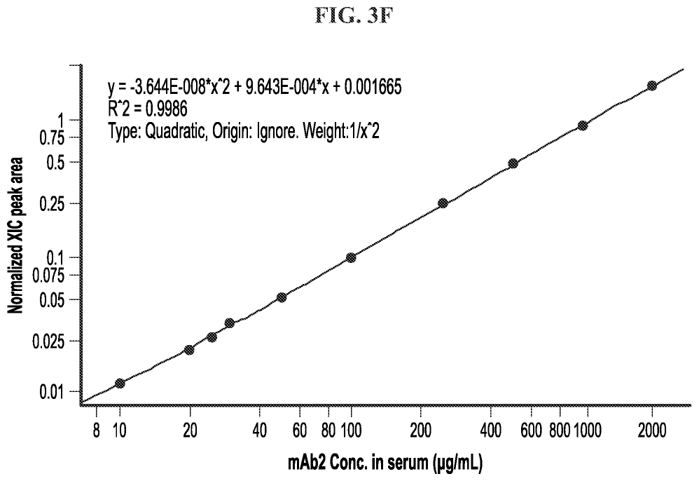

Linearity refers to the proportionality of the instrument response to the standard concentrations with the appropriate statistical model of linear or non-linear regression. In this LC-MRM-MS assay, linearity was determined by the normalized extracted ion chromatogram (XIC) peak areas of nine non-zero standards for mAb1 and ten non-zero standards for mAb2 over three days. Representative XICs of the surrogate peptides and IS peptides from the calibration standards, as well as the calibration curves for the two antibody drugs, are shown in FIG. 3. The back-calculated drug concentrations of the calibration standards using the normalized responses and the respective standard curve equation were used to estimate the accuracy of the standards using the following equation.

Accuracy % (% ACC)=100%×(measured concentration/nominal concentration).

The statistical profile of the measured concentrations of the non-zero standards for both drugs from three independent experiments are summarized in Table 3 and Table 4. The average % ACC values of the all the standards ranged from 89% to 105% for mAb1, and 92% to 106% for mAb2. The CV % (coefficient of variation) of measured concentration values for all non-zero standards varied from 2.6% to 11% for mAb1, and 0.7% to 12% for mAb2. These results met the criteria for bioanalysis that % ACC should be within ±20% of the nominal value for non-zero standards, except for standards at LLOQ or ULOQ level, which must be within ±25%; and that CV % must be ≤20% for all non-zero standards, except for standards at LLOQ or ULOQ level, which must be ≤25%.

TABLE 3
Accuracy and precision for all non-zero standards
of mAb1 from three independent experiments

| mAb1 calibration | | | | | |
|---|---|---|---|---|---|
| standard nominal | Plate 1 | Plate 2 | Plate 3 | Average % | |
| conc. (μg/mL) | ACC % | ACC % | ACC % | ACC | CV % |
| 20 | 78 | 97 | 92 | 89 | 10.8 |
| 25 | 104 | 99 | 113 | 105 | 6.9 |
| 30 | 101 | 109 | 96 | 102 | 6.3 |
| 50 | 113 | 96 | 99 | 103 | 8.7 |
| 100 | 109 | 103 | 101 | 104 | 3.8 |
| 250 | 100 | 91 | 99 | 96 | 4.8 |
| 500 | 109 | 100 | 96 | 102 | 6.4 |
| 1000 | 93 | 109 | 104 | 102 | 8.3 |
| 2000 | 101 | 96 | 99 | 99 | 2.6 |

TABLE 4
Accuracy and precision for all non-zero standards
of mAb2 from three independent experiments

| mAb2 calibration | | | | | |
|---|---|---|---|---|---|
| standard nominal | Plate 1 | Plate 2 | Plate 3 | Average % | |
| conc. (μg/mL) | ACC % | ACC % | ACC % | ACC | CV % |
| 10 | 80 | 94 | 101 | 92 | 11.6 |
| 20 | 91 | 104 | 93 | 96 | 7.4 |
| 25 | 104 | 104 | 108 | 105 | 2.1 |
| 30 | 109 | 111 | 97 | 106 | 7.3 |
| 50 | 114 | 96 | 101 | 103 | 8.9 |
| 100 | 111 | 102 | 103 | 105 | 4.5 |
| 250 | 99 | 89 | 100 | 96 | 6.1 |
| 500 | 108 | 97 | 97 | 101 | 6.0 |
| 1000 | 93 | 102 | 101 | 99 | 5.2 |
| 2000 | 102 | 101 | 100 | 101 | 0.7 |

### 2.2 Accuracy and Precision

Accuracy refers to the closeness of agreement between a measured result and its theoretical true value, and is expressed as percent accuracy (% ACC). Precision refers to the quantitative measure of the random variation between repeated measurements of the same sample, which is expressed as the percentage of coefficient of variation (CV % Conc). The intra-day accuracy and precision were determined by five replicates of qualification QCs per run, prepared as described in the Preparation of standard solutions above, in three independent measurements over three days. The inter-day accuracy and precision were determined by three independent measurements from sample preparation to LC-MS/MS analysis, each with five replicates of qualification QCs, over three days. Data for the intra-day and inter-day accuracy and precision parameters are presented in Table 5-1, Table 5-2 and Table 5-3.

TABLE 5-1
Intra-day (N = 5) accuracy and precision of mAb1 at
five QC levels for the LC-MRM-MS assay.

| | | Intra-day Replicate | | Intra-day Replicate | | Intra-day Replicate | |
|---|---|---|---|---|---|---|---|
| QC standards | | 1 (N = 5) | | 2 (N = 5) | | 3 (N = 5) | |
| | Nominal Conc. | Average | | Average | | Average | |
| Level | (μg/mL) | ACC % | Precision | ACC % | Precision | ACC % | Precision |
| LLOQ | 20 | 114 | 0.02 | 89 | 0.03 | 100 | 0.06 |
| LQC | 60 | 98 | 0.01 | 92 | 0.04 | 96 | 0.03 |
| MQC | 750 | 107 | 0.03 | 99 | 0.02 | 100 | 0.02 |
| HQC | 1500 | 107 | 0.03 | 99 | 0.03 | 100 | 0.02 |
| ULOQ | 2000 | 105 | 0.02 | 99 | 0.05 | 106 | 0.06 |

TABLE 5-2
Intra-day (N = 5) accuracy and precision of mAb2 at
five QC levels for the LC-MRM-MS assay.

| | | Intra-day Replicate | | Intra-day Replicate | | Intra-day Replicate | |
|---|---|---|---|---|---|---|---|
| QC standards | | 1 (N = 5) | | 2 (N = 5) | | 3 (N = 5) | |
| | Nominal Conc. | Average | | Average | | Average | |
| Level | (μg/mL) | ACC % | Precision | ACC % | Precision | ACC % | Precision |
| LLOQ | 10 | 111 | 0.07 | 93 | 0.06 | 107 | 0.09 |
| LQC | 30 | 100 | 0.03 | 92 | 0.02 | 96 | 0.05 |
| MQC | 750 | 107 | 0.02 | 102 | 0.02 | 102 | 0.02 |
| HQC | 1500 | 106 | 0.03 | 98 | 0.03 | 102 | 0.02 |
| ULOQ | 2000 | 101 | 0.03 | 110 | 0.04 | 106 | 0.02 |

TABLE 5-3
Inter-day (N = 3) accuracy and precision of mAb1 and mAb2
at five QC levels for the LC-MRM-MS assay.

| QC standards | | | | | | |
|---|---|---|---|---|---|---|
| | mAb1 | mAb2 | Inter-day (N = 3) | | | |
| | Nominal | Nominal | mAb1 | | mAb1 | |
| | Conc. | Conc. | Average | mAb2 | Average | mAb2 |
| Level | (μg/mL) | (μg/mL) | ACC % | Precision | ACC % | Precision |
| LLOQ | 20 | 10 | 101 | 0.13 | 104 | 0.09 |
| LQC | 60 | 30 | 95 | 0.03 | 96 | 0.04 |
| MQC | 750 | 750 | 102 | 0.04 | 103 | 0.03 |
| HQC | 1500 | 1500 | 102 | 0.04 | 102 | 0.04 |
| ULOQ | 2000 | 2000 | 103 | 0.03 | 106 | 0.04 |

The statistical profile of the inter-day accuracy and precision assessment show that % ACC values for mAb1 of all five QCs ranged from 95% to 103%, and % ACC values for mAb2 of all five QCs ranged from 96% to 106%. The inter-day CV % Conc values for all QCs varied 3% and 13% for mAb1 and 3% and 9% for mAb2. For the intra-day assessment, the % ACC values for mAb1 of the five QCs were between 89% and 114%, with CV % Conc values between 1% and 6%, and the % ACC values for mAb2 of the five QCs were between 92% and 111%, with CV % Conc values between 2% and 9%. These results demonstrated that, for both mAb1 and mAb2, both intra-day and inter-day accuracy of this LC-MRM-MS assay are between 80% and 120% for HQC, MQC, and LQC, and between 75% and 125% for LLOQ and ULOQ. Both intra-day and inter-day CV % of measured concentration are within 20% for HCQ, MQC, and LQC, and within 25% for LLOQ and ULOQ.

### 2.3 Selectivity

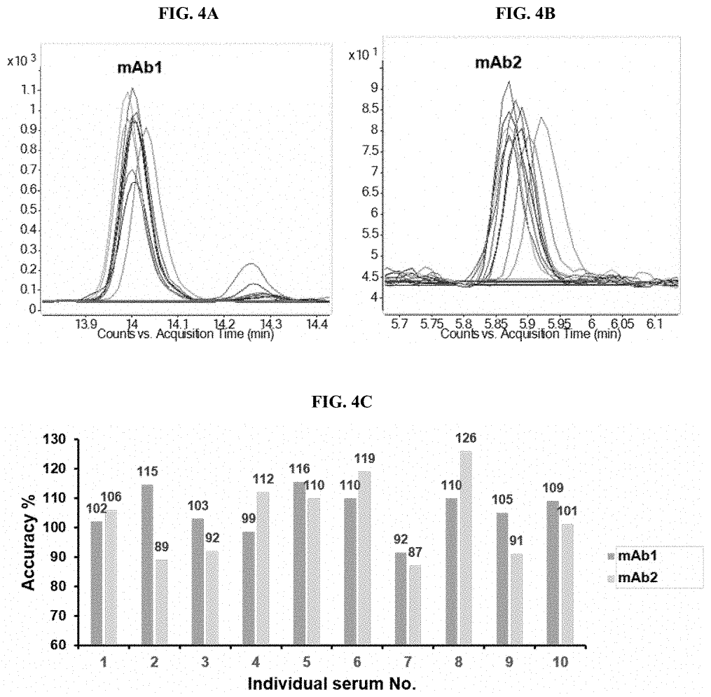

Selectivity refers to the selective and specific quantitation of the analyte in the presence of varying endogenous and non-assay-specific matrix constituents. A set of ten individual naive human serum samples were analyzed to examine if the assay was subject to non-specific matrix interference. For both mAb1 and mAb2, all samples were shown to be below the limit of quantitation (BLQ), which is 20 μg/mL for mAb1, and 10 μg/mL for mAb2. Further evidence of selectivity was evaluated by accuracy assessment of LLOQ spiked individual naive human serum samples. As shown in FIG. 4, for each of the ten individual serum samples co-spiked with 20 μg/mL of mAb1 and 10 μg/mL of mAb2, the measured concentrations of mAb1 were within ±25% of the nominal value, with % ACC values ranging from 92% to 116%. The % ACC values for mAb2 ranged from 87% to 126%, with measured concentration of mAb2 in one sample out of the ±25% of the nominal value. These results all met the acceptance criteria stated in the Bioanalytical Method Validation Guidance for Industry, that at least 80% of the LLOQ-spiked naive samples must meet the acceptance criteria of % ACC within ±25% of the nominal value (U.S. Department of Health and Human Services, Food and Drug Administration, Center for Drug Evaluation and Research, Center for Veterinary Medicine. Bioanalytical Method Validation Guidance for Industry 2018).

From these evaluations, it was demonstrated that the LC-MRM-MS assay developed is selective for human serum samples containing mAb1 and mAb2. In addition, the results obtained with the LLOQ-spiked samples confirmed that the assay in neat serum can quantitate levels of total mAb1 as low as 20 μg/mL, and total mAb2 as low as 10 μg/mL, further establishing the LLOQ for the MRM assay.

### 2.4 Specificity

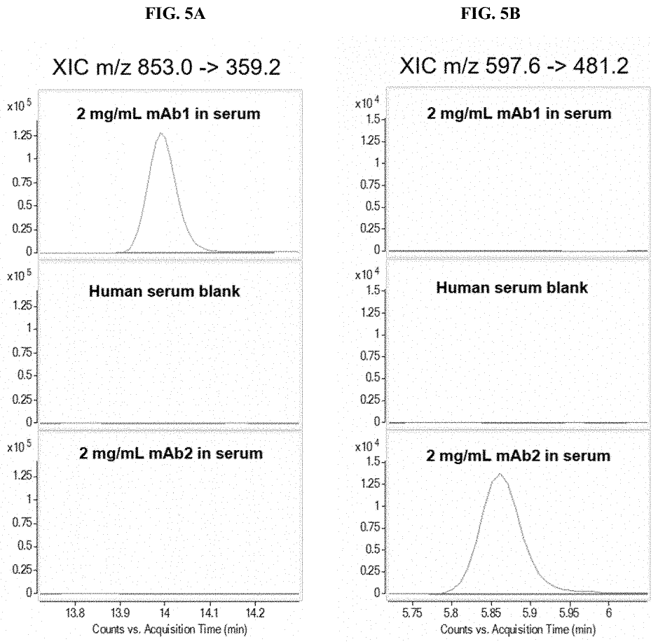

Specificity refers to the ability of the method to assess the analyte in the presence of other components that are expected to be present. Because mAb1 and mAb2 are co-administered as an antibody cocktail therapy, the interference from concomitant medication for the method specificity of each drug was evaluated. As shown in the XICs of FIG. 5, the signal from the transition channel of mAb1 surrogate peptides (m/z 853.0->m/z 359.2) at retention time of 14 minutes was not detectable in 2 mg/mL mAb2 in human serum (FIG. 5A). A similar response was observed for the signal from the transition channel of mAb2 (m/z 597.6->m/z 481.2) in the sample of 2 mg/mL mAb1 in human serum (FIG. 5B).

To systematically evaluate the drug specificity for each drug, the accuracy of QC standards made with one drug alone were compared with the accuracy of QC standards containing 2 mg/mL of the co-administered drug, at five different QC concentration levels. The % ACC values of mAb1 QCs ranged from 98% to 103% without the presence of mAb2, which was comparable to the ACC % range (97% to 120%) measured for mAb1 QCs with 2 mg/mL of mAb2 in serum (FIG. 5C). The % ACC values of mAb2 QCs ranged from 100% to 114% without the presence of mAb1, which was also comparable to the % ACC range (96% to 108%) measured for mAb2 QCs with 2 mg/mL of mAb1 spiked in serum (FIG. 5D). The % ACC values of QCs for each drug at all five QCs level, with or without the presence of the co-administered drug, met the acceptance criteria of % ACC within ±20% of the nominal value, except % ACC of ULOQ and LLOQ within ±25% of nominal value.

### 2.5 Analyte Stability

Analyte stability refers to the ability to accurately measure an analyte within the acceptance criteria of the assay after the sample has been subjected to stress or different storage conditions. To fit for the purpose of this assay, the stability of intact therapeutic protein in human serum after exposure to three freeze/thaw (FT) cycles and the stability of digested analyte during storage in the UPLC autosampler were evaluated.

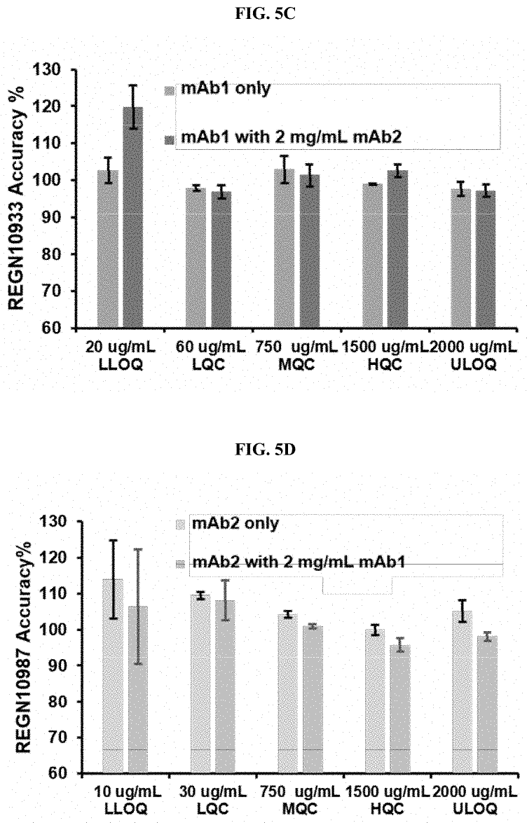

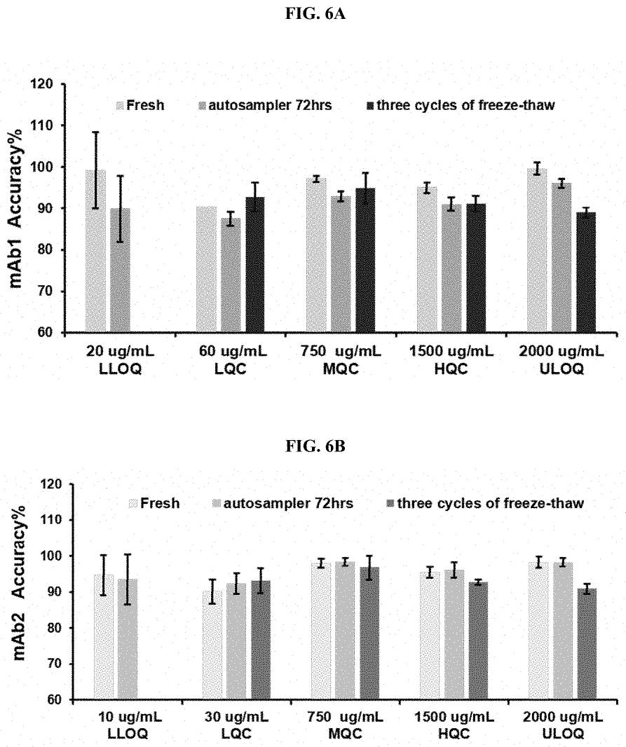

To assess analyte stability under the conditions of freeze/thaw cycles, five replicates of ULOQ, HQC, MQC, and LQC were frozen/thawed for three cycles before digestion. To assess the analyte stability in the instrument autosampler, five replicates of ULOQ, HQC, MQC, LQC and LLOQ were analyzed after 72 hours of storage at 7° C. in the autosampler. The measured accuracy of fresh QC samples was compared with that of the QC samples subjected to freeze/thaw cycles before digestion, as well as QC samples stored for long hours in autosampler after digestion. As shown in FIG. 6, no significant changes of measured accuracy of the drug concentrations were observed for the evaluated conditions. All QCs met the acceptance criteria of % ACC of all QC within ±20% of the nominal value, except % ACC of ULOQ within ±25% of the nominal value, indicating that both mAb1 and mAb2 are stable in human serum under the conditions tested and this stability could be accurately assessed using the method of the invention.

### Example 3

### Measurement of REGEN-COV Concentrations in COVID-19 Patient Serum Samples

Due to the urgent need for a reliable quantitative pharmacokinetic assay for the characterization of REGEN-COV, the developed LC-MRM-MS assay served as an interim method to determine total concentrations of mAb1 and mAb2 in serum patient samples collected from a Phase I clinical trial of REGEN-COV in outpatients with COVID-19 (Weinrich et al.). The patients participating in the double-blind clinical study were randomly assigned (1:1:1) to receive placebo, 2.4 g of REGEN-COV (1.2 g of each antibody), or 8.0 g of REGEN-COV (4 g of each antibody) by intravenous injection. Serum samples were collected from patients in the 2.4 g and 8.0 g dosage groups pre-dose, 1 hour after drug infusion, and 2, 4, 6, 14, and 28 days after drug administration, and were analyzed by the LC-MRM-MS assay of the invention.

Calibration standards, as well as QC standards at four concentration levels in triplicates (LLOQ, LQC, MQC, HQC), were digested and analyzed together with each batch of patient samples. The run acceptance criteria for the LC-MRM-MS assay were defined by the percent accuracy of non-zero calibration standards, as well as the percent accuracy and coefficients of variation of QCs. The accuracy of measured concentration of the calibration standard must be within 25% of the nominal concentration at LLOQ, and within 20% of the nominal concentration at all other concentrations. At least two thirds of the measured QC concentrations must be within 20% or 25% (LLOQ) of their respective nominal values. At least two replicates of the QCs at each level should be within 20% or 25% (LLOQ) of their nominal concentrations. Acceptance criteria for precision is determined at each concentration level and should be within 20%. All the pre-dosage patient serum samples measured had responses below LLOQ for both mAb1 and mAb2, which further validated the selectivity of this LC-MRM-MS assay.

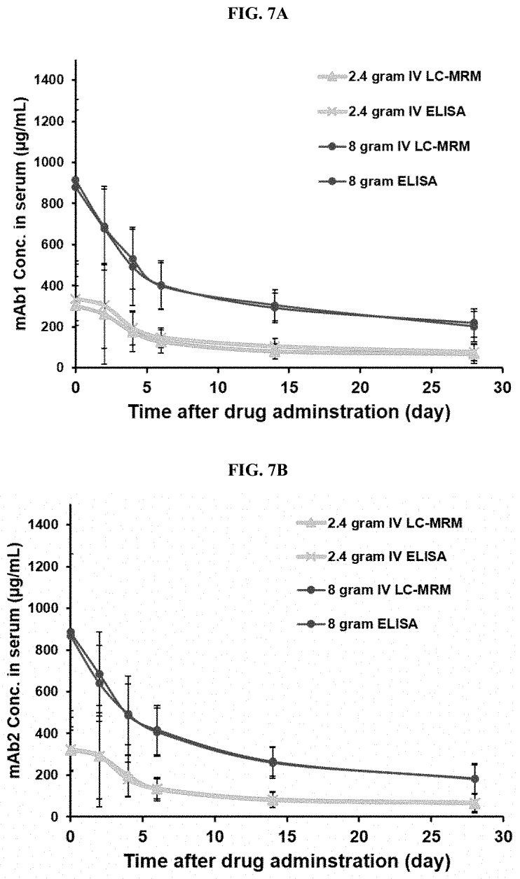

The time profile of serum drug concentration after a single dose injection was used to determine the pharmacokinetic parameters of mAb1 and mAb2, as shown in FIG. 7. The results showed that the antibody drug concentration reaches a maximum within 1 hour of intravenous injection and decays over time. The pharmacokinetics of each antibody were linear and dose-proportional, and the drug concentration in serum at Day 29 remained above the predicted neutralization target concentration based on in vitro and preclinical data (Hansen et al.; Baum et al.; Weinrich et al.). The linearity range of the developed assay covers all the clinical samples from both dosage groups and six PK sampling time points over one month after drug infusion.

### Example 4

### Method Validation by Immunoassay

Immunoassays traditionally have been the method of choice for the determination of antibody drug concentration in serum matrix for clinical sample analysis. Immunoassays measure a protein target as an intact molecule, instead of measuring surrogate fragments derived from protease digestion as in the LC-MRM-MS assay. The most critical reagents for clinical PK immunoassay development are the anti-idiotypic antibodies that specifically bind to the idiotypes of the antibody drugs, and it normally takes several months to screen and produce these reagents. Immunoassays typically provide very good sensitivity in the ng-mL range, which could not be reached by conventional LC-MRM assay without additional immunoaffinity enrichment steps. Another advantage of the immunoassay is that a large batch of patient sample analysis can be carried out in a high-throughput format once the method is established.

After an electrochemiluminescence immunoassay for REGEN-COV was fully validated, the Phase I clinical samples were reanalyzed with this method. Results were compared between measured concentrations by immunoassay and by the LC-MRM-MS method of the invention, as shown in FIG. 7. The comparison indicated that there is good agreement between the results of both assays. The differences of average drug concentrations from individual patients (n=15˜20 per dose group) measured by the two assays were within 23% for mAb1, and 11% for mAb2. This validated that the LC-MRM-MS method of the invention is effective for accurate, sensitive and specific measurement of antibody therapeutics, while having the advantage of faster development compared to an immunoassay that relies on an anti-idiotypic antibody reagent.

What is claimed is:

1. A method for simultaneously quantitating at least two therapeutic proteins, comprising:
(a) obtaining a sample including a first therapeutic protein and a second therapeutic protein;
(b) generating at least one unique surrogate peptide for each of said first and second therapeutic proteins by contacting said sample to at least two digestive enzymes;
(c) quantitating said surrogate peptides using a mass spectrometer; and
(d) quantitating said first and second therapeutic proteins using the quantitated surrogate peptides.

2. The method of claim 1, wherein said digestive enzymes are chosen from a group consisting of trypsin, chymotrypsin, LysC, LysN, AspN, GluC and ArgC.

3. The method of claim 1, wherein said digestive enzymes are trypsin and AspN.

4. The method of claim 1, wherein said first and second therapeutic proteins are selected from a group consisting of an antibody, a monoclonal antibody, a bispecific antibody, an antibody fragment, a Fab region of an antibody, an antibody-drug conjugate, or a fusion protein.

5. The method of claim 1, wherein said first therapeutic protein is casirivimab and said second therapeutic protein is imdevimab.

6. The method of claim 1, wherein said mass spectrometer is an electrospray ionization mass spectrometer, nano-electrospray ionization mass spectrometer, or a triple quadrupole mass spectrometer.

7. The method of claim 1, wherein said mass spectrometer is coupled to a chromatography system.

8. The method of claim 7, wherein said chromatography system comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

9. The method of claim 1, wherein said sample includes human serum.

10. The method of claim 1, further comprising selecting said digestive enzymes using in silico analysis of potential surrogate peptides.

11. The method of claim 1, further comprising administering said first and second therapeutic proteins to a subject.

12. The method of claim 1, wherein said method has a dynamic range of about 10 to about 2000 μg/mL of the first therapeutic protein in the sample.

13. The method of claim 1, wherein said method has a dynamic range of about 10 to about 2000 μg/mL of the second therapeutic protein in the sample.

14. The method of claim 1, wherein said mass spectrometer is capable performing a multiple reaction monitoring or parallel reaction monitoring.

15. The method of claim 1, further comprising the steps of conducting peptide mapping of said surrogate peptides, selecting unique peptides and fragment ions of the surrogate peptides to generate multiple reaction monitoring transitions, selecting the top two or top three transitions of the surrogate peptides, optimizing collision energy of the surrogate peptides, subsequently generating a calibration curve, and determining a LLOQ (lower limit of quantification) according to the calibration curve.

16. The method of claim 1, further comprising selecting said at least one surrogate peptide specific to said first or said second therapeutic protein, wherein the at least one surrogate peptide is pre-selected by determining that:
i. the surrogate peptide is specific to a digest of the therapeutic protein to be quantified;
ii. the surrogate peptide is specifically absent from the protease digest of the preparation in the absence of the at least one therapeutic protein;
iii. the surrogate peptide produces a strong signal in a mass spectrometric analysis; and
iv. the surrogate peptide produces a distinguishable signal in a mass spectrometric analysis.

17. A method for simultaneously quantitating casirivimab and imdevimab from an administered antibody cocktail, comprising:
(a) obtaining a serum sample including casirivimab and imdevimab;
(b) generating at least one unique surrogate peptide for each of casirivimab and imdevimab by contacting said sample to trypsin and AspN;
(c) quantitating said surrogate peptides using a mass spectrometer; and
(d) quantitating casirivimab and imdevimab using the quantitated surrogate peptides.

18. The method of claim 17, wherein said surrogate peptides comprise the amino acid sequences LLIYAASNLETGVPSR and DTAVYYCASGS.

19. The method of claim 17, wherein said mass spectrometer is an electrospray ionization mass spectrometer, nano-electrospray ionization mass spectrometer, or a triple quadrupole mass spectrometer.

20. The method of claim 17, wherein said mass spectrometer is coupled to a liquid chromatography system.

21. The method of claim 17, further comprising administering casirivimab and imdevimab to a subject.

22. The method of claim 17, wherein said mass spectrometer is capable performing a multiple reaction monitoring or parallel reaction monitoring.
